(19) United States
(12) Patent Application Publication
(10) Pub. No.: US 2025/0193572 A1
(43) Pub. Date: Jun. 12, 2025

(54) EAR-WEARABLE ELECTRONIC DEVICE INCLUDING RECEIVER CABLE WITH AN OPTICAL SENSOR

(71) Applicant: Starkey Laboratories, Inc., Eden Prairie, MN (US)

(72) Inventors: Andy S. Lin, Eden Prairie, MN (US); Kyle D. Olson, St. Louis Park, MN (US); Mark J. Schroeder, Eden Prairie, MN (US)

(21) Appl. No.: 18/973,900

(22) Filed: Dec. 9, 2024

Related U.S. Application Data
(60) Provisional application No. 63/608,914, filed on Dec. 12, 2023.

(51) Int. Cl. H04R 1/10; A61B 5/024; H04R 25/00

(52) U.S. Cl. CPC H04R 1/1041; A61B 5/02433; H04R 25/556; H04R 25/602; H04R 2225/025; H04R 2225/61; H04R 2460/03

(57) ABSTRACT

A receiver-in-canal (RIC) hearing device comprises a housing configured for deployment behind an ear of a user of the hearing device. A power source, audio components, a communication device, and a controller are respectively disposed in the housing. A cable assembly comprises a cable, a receiver disposed at a distal end of the cable, and a connector disposed at a proximal end of the cable and configured to attach to the housing. An optical sensor is disposed in the connector and configured to generate a proximity signal based on proximity of the optical sensor to skin at or near the user's ear. The controller is configured to operate the hearing device in a nominal power mode in response to the proximity signal exceeding a threshold and to operate the hearing device in a low power mode in response to the proximity signal falling below the threshold.

### CROSS REFERENCE TO RELATED APPLICATION

This application claims the benefit of 63/608,914 filed Dec. 12, 2023, the disclosure of which is incorporated by reference herein in its entirety.

### TECHNICAL FIELD

This application relates generally to ear-level electronic devices and systems, and, in particular, to hearing devices which include a receiver cable.

### SUMMARY

Some embodiments are directed to a receiver-in-canal (RIC) hearing device comprising a housing configured for deployment behind an ear of a user of the hearing device. A power source, audio components, a communication device, and a controller are respectively disposed in the housing. A cable assembly comprises a cable, a receiver disposed at a distal end of the cable, and a connector disposed at a proximal end of the cable and configured to attach to the housing. An optical sensor is disposed in the connector and configured to generate a proximity signal based on proximity of the optical sensor to skin at or near the user's ear. The controller is configured to operate the hearing device in a nominal power mode in response to the proximity signal exceeding a threshold and to operate the hearing device in a low power mode in response to the proximity signal falling below the threshold.

In some embodiments, the controller is configured to operate the hearing device in the nominal power mode in response to the proximity signal exceeding an On-threshold, and operate the hearing device in the low power mode in response to the proximity signal falling below an Off-threshold lower than the On-threshold. In other embodiments, the controller is configured to operate the hearing device in the nominal power mode in response to the proximity signal exceeding an On-threshold, operate the hearing device in the low power mode in response to the proximity signal falling below an Off-threshold lower than the On-threshold, and interpret changes of the proximity signal below a User Control threshold due to user movement of the ear or the housing as a user input for controlling the hearing device, wherein the User Control threshold is between the On-threshold and the Off-threshold and is active after the proximity signal exceeds the On-threshold.

Some embodiments are directed to a cable assembly configured to attach to a housing of a RIC hearing device. The cable assembly comprises a cable, a receiver disposed at a distal end of the cable, and a connector disposed at a proximal end of the cable and configured to attach to the housing of the hearing device. The connector comprises an optical sensor configured to generate a proximity signal based on proximity of the optical sensor to skin at or near an ear of a user of the hearing device, an interface configured to communicatively couple the optical sensor and a controller of the hearing device, and power and ground contacts configured to couple with power and ground contacts of the hearing device housing, wherein the connector comprises material substantially transparent to light generated by the optical sensor.

The above summary is not intended to describe each disclosed embodiment or every implementation of the present disclosure. The figures and the detailed description below more particularly exemplify illustrative embodiments.

### BRIEF DESCRIPTION OF THE DRAWINGS

Throughout the specification reference is made to the appended drawings wherein:

FIG. 1 illustrates a representative RIC hearing device in accordance with any of the embodiments disclosed herein.

FIG. 2 illustrates a representative RIC hearing device in accordance with any of the embodiments disclosed herein.

FIG. 3 illustrates elements of a RIC cable connector and a housing connector in accordance with any of the embodiments disclosed herein.

FIG. 4 illustrates an embodiment of an optical sensor in accordance with any of the embodiments disclosed herein.

FIG. 5 illustrates an embodiment of an optical sensor in accordance with any of the embodiments disclosed herein.

FIG. 6 is a state diagram that illustrates various functions performed by a hearing device when implementing an automatic On/Off feature in accordance with any of the embodiments disclosed herein.

FIG. 7 graphically illustrates a user control feature implemented by a hearing device in accordance with any of the embodiments.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Embodiments disclosed herein are directed to any ear-wearable or ear-level electronic device which includes a receiver cable without departing from the scope of this disclosure. Some embodiments are directed to hearing devices that can aid a person with impaired hearing. In particular, embodiments of the disclosure are directed to a RIC hearing device which includes a RIC cable connected to an earpiece. An acoustic transducer (e.g., a receiver) is disposed in the earpiece. Throughout this disclosure, reference is made to an ear-wearable electronic device in the form of a hearing device, which is understood to refer to a system comprising a single left ear device, a single right ear device, or a combination of a left ear device and a right ear device.

Battery life is always a concern for hearing device users. The power button on conventional hearing devices creates a potential entry point for contaminants and increases the likelihood of contaminant ingress, which reduces the reliability of the device. An automatic On/Off function that turns on the hearing device only when the device is on the user's ear is a highly desirable feature. The RIC cable on a standard RIC hearing device (e.g., RIC hearing aid) provides a unique opportunity to enable an automatic On/Off function without changing the housing design of the hearing device.

Currently, most hearing devices require the user to actively press and hold a power button to turn the device off. One approach to automating this process is to utilize a motion sensor (e.g., an inertial measurement unit or IMU) to turn off the device after a period of inactivity. However, it is undesirable to use this approach to turn the device back on due to the high power requirement to keep monitoring for a change in condition. It is also undesirable to use this feature when turning the device off because it takes a few minutes before turning off, thus wasting power. Moreover, users may not perceive that the powering off feature is working correctly when the device is removed from the user's ear.

Moreover, utilizing an IMU to automatically turn a hearing device on and off cannot address the scenario where hearing devices are placed in a pocket or purse, as the IMU will continue to experience motion even though the hearing devices are not being worn. One possible improvement is to add an optical sensor in the housing of the hearing device.

However, this approach would require a change in hardware design involving mounting the optical sensor to the flexible printed circuit board of the device. Also, a window would have to be added on the hearing device housing to allow the optical sensor to carry out its detection function, which adds steps and cost to the manufacturing process.

Embodiments of the disclosure are defined in the claims. However, below there is provided a non-exhaustive listing of non-limiting examples. Any one or more of the features of these examples may be combined with any one or more features of another example, embodiment, or aspect described herein.

Example Ex1. A receiver-in-canal (RIC) hearing device comprises a housing configured for deployment behind an ear of a user of the hearing device. A power source, audio components, a communication device, and a controller are respectively disposed in the housing. A cable assembly comprises a cable, a receiver disposed at a distal end of the cable, and a connector disposed at a proximal end of the cable and configured to attach to the housing. An optical sensor is disposed in the connector and configured to generate a proximity signal based on proximity of the optical sensor to skin at or near the user's ear. The controller is configured to operate the hearing device in a nominal power mode in response to the proximity signal exceeding a threshold and to operate the hearing device in a low power mode in response to the proximity signal falling below the threshold.

Example Ex2. The hearing device according to Ex1, wherein the controller operates the hearing device in the low power mode in response to the proximity signal falling below the threshold for a specified duration of time.

Example Ex3. The hearing device according to Ex1 or Ex2, comprising a motion sensor disposed in the housing, wherein the controller is configured to operate the hearing device in the low power mode in response to the proximity signal falling below the threshold and the motion sensor indicating an absence of housing motion for a specified duration of time.

Example Ex4. The hearing device according to any one of Ex1-3, wherein the audio components communicate a first output to the receiver in response to the proximity signal exceeding the threshold and communicates a second audio output or no audio output to the receiver in response to the proximity signal falling below the threshold.

Example Ex5. The hearing device according to any one of Ex1-4, wherein the connector comprises an interface configured to communicatively couple the optical sensor and the controller, and power and ground contacts arranged to couple with power and ground contacts of the housing.

Example Ex6. The hearing device according to any one of Ex1-5, wherein the optical sensor is configured to sense a heart rate of the user.

Example Ex7. The hearing device according to any one of Ex1-6, wherein the optical sensor is configured to sense blood oxygen saturation of the user.

Example Ex8. The hearing device according to any one of Ex1-7, wherein the optical sensor comprises an infrared (IR) emitter and a photodetector sensitive to IR light, and the connector comprises material substantially transparent to IR light.

Example Ex9. The hearing device according to any one of Ex1-7, wherein the optical sensor comprises an infrared (IR) emitter, one or more visible light emitters, photodetectors sensitive to IR light and one or more visible light wavelengths, and the connector comprises material substantially transparent to IR light and the one or more visible light wavelengths.

Example Ex10. The hearing device according to Ex9, wherein the controller is configured to control the one or more visible light emitters as visible indicators to the user.

Example Ex11. The hearing device according to any one of Ex1-10, wherein the optical sensor operates at a first update rate when in the low power mode and a second update rate when in the nominal power mode, wherein the second update rate is faster than the first update rate.

Example Ex12. The hearing device according to any one of Ex1-11, wherein the power source comprises a lithium-ion battery coupled to a charge pump, a buck converter, a boost converter, or a buck/boost converter.

Example Ex13. The hearing device according to any one of Ex1-11, wherein the power source comprises a zinc-air battery coupled to a charge pump or a boost converter, and the optical sensor is coupled to the charge pump or the boost converter.

Example Ex14. The hearing device according to any one of Ex1-13, wherein the controller is configured to interpret changes of the proximity signal above the threshold due to user movement of the ear or the housing as a user input for controlling the hearing device.

Example Ex15. The hearing device according to Ex14, wherein the controller is configured to interpret different durations of proximity signal changes as different user inputs.

Example Ex16. The hearing device according to Ex15, wherein the different durations of proximity signal changes comprise a short duration change and a long duration change.

Example Ex17. The hearing device according to Ex15, wherein the different durations of proximity signal changes comprise a short duration change, a medium duration change, and a long duration change.

Example Ex18. The hearing device according to any one of Ex1-17, comprising an additional optical sensor disposed in the receiver and configured to generate an additional proximity signal based on proximity of the additional optical sensor to skin of the user's ear canal, wherein the controller is configured to operate the hearing device in the nominal power mode in response to the proximity signal and the additional proximity signal exceeding the threshold and to operate the hearing device in a low power mode in response to the proximity signal and the additional proximity signal falling below the threshold.

Example Ex19. The hearing device according to Ex1, wherein the controller is configured to operate the hearing device in the nominal power mode in response to the proximity signal exceeding an On-threshold, and operate the hearing device in the low power mode in response to the proximity signal falling below an Off-threshold lower than the On-threshold.

Example Ex20. The hearing device according to Ex1, wherein the controller is configured to operate the hearing device in the nominal power mode in response to the proximity signal exceeding an On-threshold, operate the hearing device in the low power mode in response to the proximity signal falling below an Off-threshold lower than the On-threshold, and interpret changes of the proximity signal below a User Control threshold due to user movement of the ear or the housing as a user input for controlling the hearing device, wherein the User Control threshold is between the On-threshold and the Off-threshold and is active after the proximity signal exceeds the On-threshold.

Example Ex21. A cable assembly is configured to attach to a housing of a receiver-in-canal (RIC) hearing device, the cable assembly comprising a cable, a receiver disposed at a distal end of the cable, and a connector disposed at a proximal end of the cable and configured to attach to the housing of the hearing device. The connector comprises an optical sensor configured to generate a proximity signal based on proximity of the optical sensor to skin at or near an ear of a user of the hearing device, an interface configured to communicatively couple the optical sensor and a controller of the hearing device, and power and ground contacts configured to couple with power and ground contacts of the hearing device housing, wherein the connector comprises material substantially transparent to light generated by the optical sensor.

Example Ex22. The cable assembly according to Ex21, wherein the optical sensor is configured to sense a heart rate of the user.

Example Ex23. The cable assembly according to Ex21 or Ex22, wherein the optical sensor is configured to sense blood oxygen saturation of the user.

Example Ex24. The cable assembly according to any one of Ex21-23, wherein the optical sensor comprises an infrared (IR) emitter and a photodetector sensitive to IR light, and the connector comprises material substantially transparent to IR light.

Example Ex25. The cable assembly according to any one of Ex21-23, wherein the optical sensor comprises an infrared (IR) emitter, one or more visible light emitters, photodetectors sensitive to IR light and one or more visible light wavelengths, and the connector comprises material substantially transparent to IR light and the one or more visible light wavelengths.

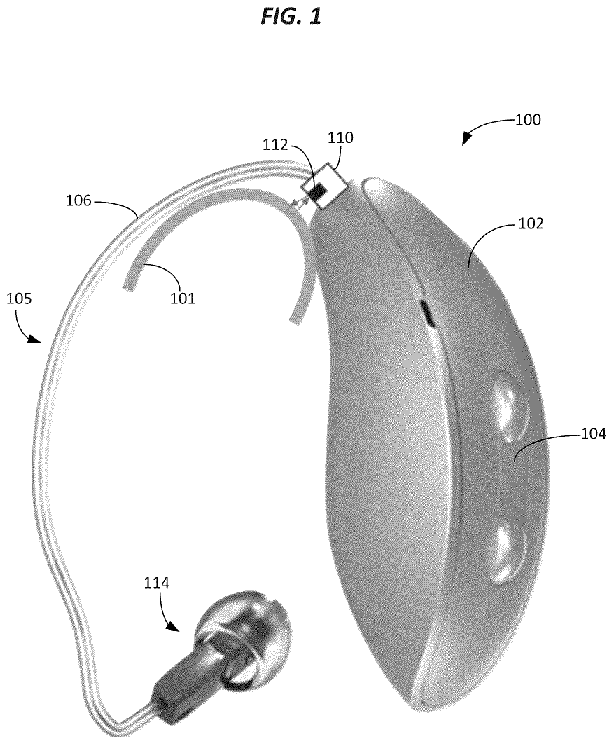

FIG. 1 illustrates a representative RIC hearing device in accordance with any of the embodiments disclosed herein. The RIC hearing device 100 includes a housing 102 within which various components are disposed. The housing 102 is configured to be positioned behind the ear 101 of the user. A switch actuator 104 (e.g., a rocker switch) is supported by the housing 102 and manipulatable by the user (e.g., for changing the volume).

The hearing device 100 comprises a cable assembly 105 which includes a cable 106 having a proximal end and a distal end. An earpiece 114 is disposed at the distal end of the cable 106 and includes an acoustic transducer in the form of a receiver. A connector 110 is disposed at the proximal end of the cable 106 and configured to facilitate attachment of the cable assembly 105 to the housing 102. An optical sensor 112 is disposed in the connector 110. The optical sensor 112 is configured to sense proximity of the connector 110 to skin at or near the user's ear 101.

The optical sensor 112 operates by emitting a beam of light (e.g., infrared light) and measuring the intensity of reflected light to determine the proximity of the connector 110 to the user's ear 101. The optical sensor 112 operates as a reflective sensor that relies on reflection of the emitted light from a reflective surface, such as the user's ear. When the connector 110 is close to the user's ear, the intensity of the reflected light from the user's ear as measured by the optical sensor 112 increases (e.g., is high). When the connector 110 is moved away from the user's ear, the intensity of the reflected light from the user's ear is reduced (e.g., is low).

The connector 110 comprises material substantially transparent to light generated by the optical sensor 112. For example, the optical sensor 112 can be configured to emit infrared light, and the connector 110 comprises material substantially transparent to infrared light. By way of further example, the optical sensor 112 can be configured to emit infrared light and visible light, and the connector 110 comprises material substantially transparent to infrared light and visible light. Suitable materials for the connector 110 include IR transparent or clear acrylic, polycarbonate, polyamide, and silicone, among others.

The optical sensor 112 generates a proximity signal which has a magnitude that corresponds to the proximity of the connector 110 to the user's ear. A threshold of the proximity signal can be established to determine when the hearing device 100 is resting behind the user's ear and when the hearing device 100 is moved away from the user's ear (e.g., removed from the ear). By comparing the proximity signal to the threshold, a controller of the hearing device 100 can automatically turn power off when the hearing device 100 is removed from the user's ear, and turn power on when the hearing device 100 is placed behind the user's ear.

Use of the optical sensor 112 allows the automatic On/Off feature of the hearing device 100 to be implemented with low power consumption. By placing the optical sensor 112 in the connector 110 of the cable 106, changes to the design of the housing 102 can be avoided. Incorporating the optical sensor 112 in the connector 110 rather than in the housing 102 provides the opportunity to retrofit older hearing devices to enable the automatic On/Off feature. Moreover, the optical sensor 112 may be upgraded (e.g., with newer components) without any changes being made to the housing 102 or its components. Also, moving the optical sensor 112 from the housing 102 to the connector 110 provides for the reduction in the size of the housing 102. It is noted that, with the placement of the optical sensor 112 in the connector 110 and the bend in the cable 106, it is very unlikely that the optical sensor 112 will be triggered in concerning situations such as when the hearing device 100 is placed in a pocket or in a purse.

One known issue with wearing a RIC hearing device is interference with the arms of eyeglasses. If the optical sensor 112 is incorporated in the housing 102 rather than the connector 110, the eyeglasses have the potential of pushing the housing 102 up and falsely turning off the hearing device 100 if the separation exceeds the proximity signal threshold.

This scenario would not occur in a configuration in which the optical sensor 112 is incorporated in the connector 110 because at this location, it is more likely for the eyeglasses to push the connector 110 down or to the side instead of upward.

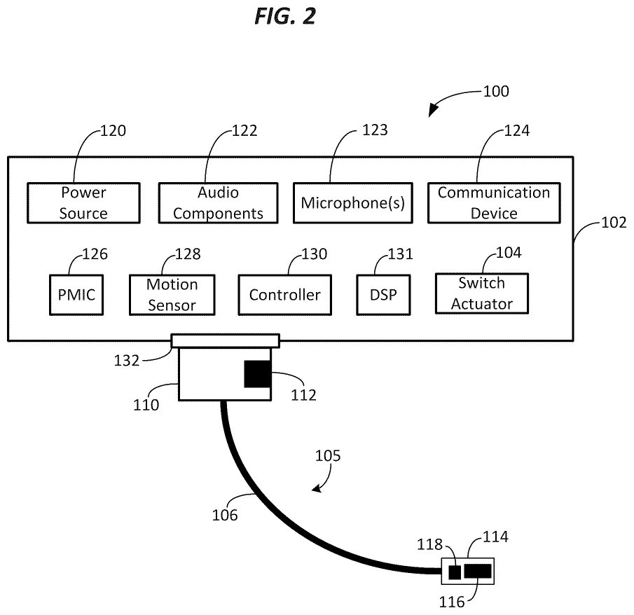

FIG. 2 illustrates a representative RIC hearing device in accordance with any of the embodiments disclosed herein. The RIC hearing device 100 includes a housing 102 within which various components are disposed and is configured to rest behind the user's ear. The hearing device 100 includes a power source 120, which can be a rechargeable power source or a non-rechargeable power source. For example, the power source 120 can comprise a lithium-ion battery, which can be coupled to a charge pump, a buck converter, a boost converter, or a buck/boost converter. The power source 120 can comprise a zinc-air battery, which can be coupled to a charge pump or a boost converter. A PMIC (power management integrated circuit) 126 is coupled to the power source 120 and power consuming components of the hearing device 100. In the case of a rechargeable power source 120, the PMIC 126 cooperates with a charging unit to manage charging of the power source 120.

The hearing device 100 includes audio components 122. The audio components 122 include audio signal processing circuitry, which includes an analog front end coupled to one or more microphones 123. A communication device 124 is provided for establishing communication between the hearing device 100 and an external electronic device or system (e.g., a smartphone, tablet, wireless access point). For example, the communication device 124 can include one or more radios that conform to an IEEE 802.11 (e.g., WiFi®) or Bluetooth® (e.g., BLE, Bluetooth®) specification. It is understood that the hearing device 100 can employ other radios, such as a 900 MHz radio. In addition, the hearing device 100 can include a near-field magnetic induction (NFMI) sensor (e.g., an NFMI transceiver coupled to a magnetic antenna) for effecting short-range communications (e.g., ear-to-ear communications, ear-to-kiosk communications).

The hearing device 100 can include a motion sensor 128 which can include one or more sensors configured to sense motion and/or a position (e.g., physical state and/or activity status) of the user of the hearing device 100. The motion sensor 128 can comprise one or more of an IMU, an accelerometer(s), a gyroscope(s), and a nine-axis motion sensor. The IMU can be of a type disclosed in commonly owned U.S. Pat. No. 9,848,273, which is incorporated herein by reference.

A controller 130 of the hearing device 100 can include one or more processors or other logic devices. For example, the controller 130 can be representative of any combination of one or more logic devices (e.g., multi-core processor, microprocessor, programmable controller, general-purpose processor, special-purpose processor, hardware controller, software controller, a combined hardware and software device) and/or other digital logic circuitry (e.g., ASICs, FPGAs). The controller 130 can incorporate or be coupled to memory. The memory can include one or more types of memory, including ROM, RAM, SDRAM, NVRAM, EEPROM, and FLASH, for example. A DSP 131 can be coupled or integral to the controller 130.

A switch actuator 104, which can be implemented as a rocker switch, is supported by the housing 102 and manipulatable by the user. For example, the switch actuator 104 can be manipulated to change the volume of sound produced by the hearing device 100. As will be discussed below, a user control feature of the hearing device 100 can be implemented to supplement or replace the switch actuator 104.

The hearing device 100 comprises a cable assembly 105 which includes a cable 106. An earpiece 114 is disposed at the distal end of the cable 106 and includes a receiver 116. A wire extends along the length of the cable 106 and couples the receiver 116 to audio components 122 in the housing 102 via a cable connector 110 and a housing connector 132. The cable connector 110 is disposed at the proximal end of the cable 106 and configured to facilitate attachment of the cable assembly 105 to the housing connector 132. An optical sensor 112 is disposed in the connector 110 and is configured to sense proximity of the connector 110 to skin at or near the user's ear 101.

In some implementations, the earpiece 114 can include an additional optical sensor 118 configured to generate an additional proximity signal based on proximity of the additional optical sensor 118 to skin of the user's ear canal. The additional proximity signal generated by the additional optical sensor 118 in the earpiece 114, in combination with the proximity signal generated by the optical sensor 112 in the connector 110, can reduce the probability of falsely triggering the automatic On/Off feature of the hearing device. It is noted that additional sensor 118 can be implemented as an electrical contact sensor.

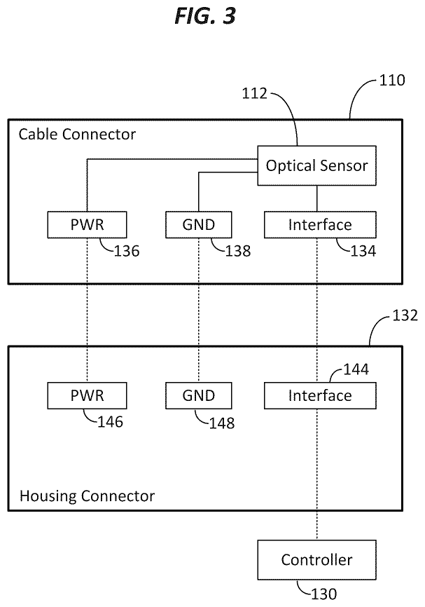

FIG. 3 illustrates elements of a cable connector 110 and a housing connector 132 in accordance with any of the embodiments disclosed herein. To supply power to the optical sensor 112, the cable connector 110 includes power and ground contacts 136, 138 which connect to power and ground contacts 146, 148 of the housing connector 132 when the cable connector 110 is attached to the housing connector 132. The power and ground contacts 146, 148 of the housing connector 132 are coupled to the power source 120 in the housing 102 (see FIG. 2).

The optical sensor 112 is coupled to the controller 130 disposed in the housing 102 via interfaces 134, 144 of the cable and housing connectors 110, 132, respectively. The controller 130 can communicate signals to the optical sensor 112 to control its operation, including powering on and powering off the optical sensor 112. When powered on, the optical sensor 112 pulses its emitter(s) and measures the reflections for determining the proximity of the optical sensor 112/cable connector 110 to a surface, such as the user's ear. The optical sensor 112 generates a proximity signal which is communicated to the controller 130 via the interfaces 134, 144.

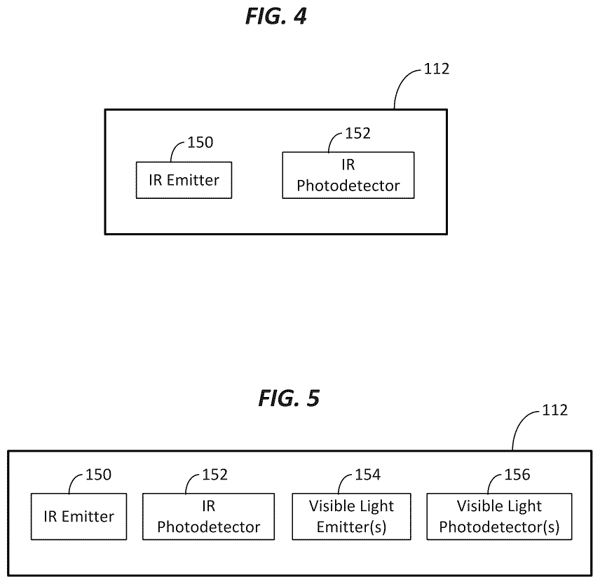

FIG. 4 illustrates an embodiment of the optical sensor 112 in accordance with any of the embodiments disclosed herein. The optical sensor 112 shown in FIG. 4 is sensitive to infrared (IR) light. The optical sensor 112 includes an IR emitter 150 (e.g., an IR LED) and a photodetector 152 (e.g., a photodiode or phototransistor) sensitive to IR light. The IR emitter 150 emits a beam of infrared light, and the photodetector 152 measures the intensity of infrared light reflected from a surface, such as the user's ear. The photodetector 152 generates an electrical signal of high magnitude in response to measuring light reflected from the user's ear, such as when the hearing device 100 is resting behind the user's ear. The photodetector 152 generates an electrical signal of low or insubstantial magnitude in response to the absence of light reflected from the user's ear, such as when the hearing device 100 has been removed from the user's ear. The magnitude of the electrical signal generated by the photodetector 152 corresponds to the proximity of the optical sensor 112 to the user's ear.

FIG. 5 illustrates an embodiment of the optical sensor 112 in accordance with any of the embodiments disclosed herein. The optical sensor 112 shown in FIG. 5 includes an IR emitter 150 and an IR photodetector 152 as previously described with reference to FIG. 4. In addition, the optical sensor 112 shown in FIG. 5 includes one or more visible light emitters 154 and one or more visible light photodetectors 156. For example, the visible light emitters 154 can include a green LED and a red LED. The visible light photodetectors 156 can include a photodetector sensitive to green light and a photodetector sensitive to red light. The optical sensor 112 can be implemented as a multifunctional optical sensor. A suitable multifunctional optical sensor is the MAXM86161 integrated optical module available from Analog Devices, Inc.

The optical sensor 112 shown in FIG. 5 can be implemented as a PPG (photoplethysmogram) sensor capable of measuring heart rate, heart rate variability (HRV), and blood oxygen saturation (SpO2). For example, the IR emitter/photodetector 150/152 and a red emitter/photodetector 154/156 can be used to provide an SpO2 measurement. A green emitter/photodetector 154/156 can be used to measure heart rate and HRV. By positioning the optical sensor 112 in the connector 110 of the cable 106, the optical sensor 112 is advantageously positioned at the back of the ear which is the best place for making physiologic measurements due to the capillary bed density and high blood perfusion.

In some implementations, the visible light emitter or emitters 154 can be used as visual indicators to communicate hearing device status information to the user, such as programming status, Bluetooth® connectivity status, and power status. The controller 130 can control the visible light emitter or emitters 154 to communicate various types of information to the user.

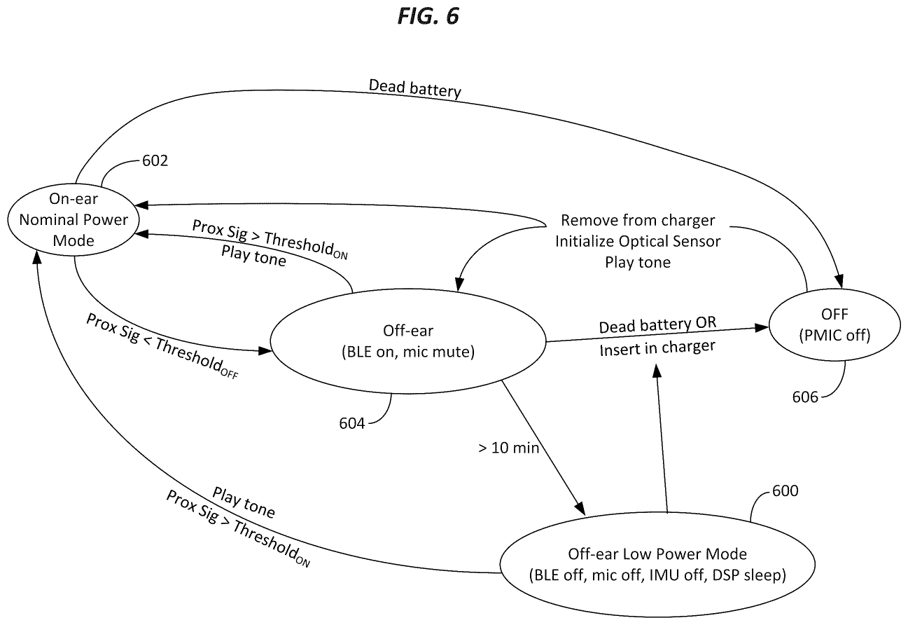

FIG. 6 is a state diagram that illustrates various functions performed by the hearing device 100 when implementing an automatic On/Off feature in accordance with any of the embodiments disclosed herein. The functionality shown in the state diagram can be implemented in firmware of the controller 130 of the hearing device 100. The state diagram includes four states: an off-ear low power mode state 600, an off-ear state 602, an on-ear nominal power mode state 604, and an off state 606. A change of state between states 600, 602, 604, 606 can be communicated to the user by different types of audio output, such as different tones or the absence of a tone. The audio output can be programmable for each state change and can include, for example, a power down tone, a bootup tone, no tone, a ding, a beep, or other sound. In some implementations, a change of state between states 600, 602, 604, 606 can be communicated to the user by different types output, including tactile outputs (via a vibration generator) or visual outputs (via one or more LEDs).

As is shown in FIG. 6, and with reference to FIG. 2, transitioning between some of the states is dependent on a proximity signal generated by the optical sensor 112 disposed in the connector 110 of the cable 106. When the housing 102 of the hearing device 100 is resting behind the user's ear, the optical sensor 112 generates a proximity signal having a magnitude that exceeds a preestablished On-threshold. When the housing 102 is removed from the user's ear, the optical sensor 112 generates a proximity signal having a magnitude that falls below a preestablished Off-threshold which is lower than the On-threshold. It is understood that, in some implementations, a single proximity signal threshold can be used rather than separate On and Off thresholds.

For purposes of discussion, it is assumed that the hearing device 100 is operating in the off-ear low power mode (state 600). When operating in the off-ear low power mode, various components of the hearing device 100 are powered off or in sleep mode. For example, when in state 600 (off-ear low power mode), the DSP 131 operates in sleep mode and the controller 130 powers down (e.g., turns off) various components of the hearing device 100, including the communication device 124 (e.g., BLE radio), the microphone(s) 123, and the motion sensor 128 (e.g., IMU). In state 600, the controller 130 causes the optical sensor 112 to periodically pulse the emitter(s) of the optical sensor 112 and measure the reflections for determining the proximity of the connector 110 to the user's ear 101. In some implementations, the optical sensor 112 operates at a first update rate when in the low power mode and a second update rate when in the nominal power mode (state 602), wherein the first update rate is slower than the second update rate.

When the proximity signal exceeds the On-threshold, the hearing device 100 transitions from state 600 to state 602, which is the on-ear nominal power mode. This state change can be accompanied by a tone being played, such as a bootup tone (e.g., a series of tones with increasing pitch). In the nominal power mode, the components and sensors of the hearing device 100 are powered on and fully functional. In state 602, the controller 130 causes the optical sensor 112 to periodically pulse the emitter(s) of the optical sensor 112 and measure the reflections for determining the proximity of the connector 110 to the user's ear 101. The hearing device 100 remains in the on-ear nominal power mode as long as the proximity signal exceeds the On-threshold and sufficient power is supplied by the power source 120 (e.g., a battery).

When operating the hearing device 100 in the nominal power mode and the proximity signal falls below the Off-threshold, the hearing device 100 transitions from state 602 to state 604, which is the off-ear state. In state 604, the radio 124 remains on and the microphone(s) is muted. In state 604, the controller 130 causes the optical sensor 112 to periodically pulse the emitter(s) of the optical sensor 112 and measure the reflections for determining the proximity of the connector 110 to the user's ear 101. If the proximity signal exceeds the On-threshold, the hearing device 100 transitions from state 604 to 602 and operates in the on-ear nominal power mode in a manner previously described. If the proximity signal remains below the Off-threshold for a predetermined duration of time (e.g., at least 10 minutes), the hearing device 100 transitions from state 604 to state 600. In some implementations, the predetermined duration of time corresponds to a time period in which no movement of the hearing device 100 is detected by the motion sensor 128 of the hearing device (e.g., at least 10 minutes of no movement). In state 600, the hearing device 100 operates in the off-ear low power mode in a manner previously described.

In case of a dead battery when operating in state 600, 602 or 604, the hearing device 100 transitions to state 606, which is the off state. In the off state, the PMIC 126 is off. It is assumed in FIG. 6 that, after reaching state 606, the user places the hearing device 100 in a charger. After charging the battery of the hearing device 100, the user removes the hearing device 100 from the charger which causes the controller 130 to initialize the optical sensor 112 and play a bootup tone. The controller 130 causes the optical sensor 112 to periodically pulse the emitter(s) of the optical sensor 112 and measure the reflections for determining the proximity of the connector 110 to the user's ear. From state 606, the hearing device 100 can transition to state 602 or 604 depending on the magnitude of the proximity signal. If the proximity signal exceeds the On-threshold, the hearing device 100 transitions from state 606 to state 602. If the proximity signal falls below the Off-threshold, the hearing device 100 transitions from state 606 to state 604.

According to various embodiments, the hearing device 100 can transition to, and operate in, a number of different low power modes. One or a combination of these low power modes can be implemented by the hearing device 100 to provide for an enhanced user experience and enhanced battery life. Representative examples of different low power modes include a mute mode, a low power mode, a lower power mode, an off mode, and a battery disconnect mode.

When operating in the mute mode, the microphone(s) 123 and the receiver 116 are muted, but the hearing device 100 is otherwise operating in the nominal power mode. The mute mode allows the hearing device 100 to boot up quickly in response to the proximity signal exceeding the On-threshold. The communication device 124 (e.g., BLE radio) remains operative such that connections are maintained so that various operations/functions can be implemented (e.g., updates, “find my hearing device,” etc.). The hearing device 100 may operate in the mute mode only for a predetermined period of time, after which the hearing 100 may transition to another low power mode. The predetermined period of time may be several minutes (e.g., 10 mins, 30 mins) or one or more hours (e.g., 1-24 hours).

In the low power mode, the communication device 124 is turned off and the DSP 131 is in sleep mode. The optical sensor 112 remains operational in the low power mode, waiting for the proximity signal to exceed the On-threshold, after which the hearing device transitions to the nominal power mode (communication device 124 turned on and DSP 131 in normal operating mode). In response to the proximity signal exceeding the On-threshold, the hearing device 100 boots up quickly (e.g., <1 s) since the DSP 131 is in sleep mode rather than being turned off.

When operating in the lower power mode, the optical sensor 112 is placed in an interrupt mode, the DSP 131 is turned off, and the communication device 124 is turned off. In the interrupt mode, the DSP 131 configures the optical sensor 112 to operate autonomously prior to powering down. When operating autonomously, the optical sensor 112 controls the timing of samples (e.g., measurements of the intensity of infrared light reflected from a surface), and compares the results of the sampling to the On- and Off-thresholds. When the optical sensor 112 detects that a change in operating state should occur, the optical sensor 112 toggles an interrupt line (e.g., from HI to LO or from LO to HI).

The PMIC 126 detects this toggle and powers up the DSP 131. The DSP 131 reads the most recent sample provided by the optical sensor 112 and determines if it should transition to an On mode or stay in one of the Off modes. Because the optical sensor 112 is in an interrupt mode and the DSP 131 is turned off, the hearing device 100 is slow (e.g., 1-5 s) to return to the nominal power mode (communication device 124 turned on, optical sensor 112 in normal operating mode, and DSP 131 in normal operating mode).

The hearing device 100 transitions to the off mode when the battery is at a predetermined state of charge (SOC), such as <10% SOC. In the off mode, the optical sensor 112, DSP 131, and communication device 124 are turned off.

The hearing device 100 transitions to the battery disconnect mode if the battery is at a predetermined SOC, such as <0%. The hearing device 100 also transitions to the battery disconnect mode if a short circuit or other high currents are detected. In the battery disconnect mode, the PMIC 126 transitions to its lowest power setting (e.g., all components powered down). The only way to wake up the hearing device 100 when in the battery disconnect mode is to charge the battery of the hearing device 100.

In some implementations, entering one of the different low power modes can involve use of the optical sensor 112 and the motions sensor 128 (e.g., IMU). The motion sensor 128 can sense motion of the hearing device 128 when the hearing device 100 is being worn by the user. The motion sensor 128 can sense the absence of hearing device motion, such as when the hearing device 128 is placed on a surface.

Consider the scenario where the hearing device 100 being worn by a user is removed from the ear and placed on a surface with high IR reflectivity (e.g., a white countertop). When the hearing device 100 is removed from the user's ear, the proximity signal initially falls below the Off-threshold, but increases above the On-threshold when the hearing device 100 is placed on a highly reflective surface. When placed on the highly reflective surface, the motion sensor 128 senses the absence of hearing device motion, thus recognizing that the hearing device 100 is not being worn. In this case, the hearing device 100 can transition to one of the different low power states discussed above.

In these low power states, the proximity signal produced by the optical sensor 112 can be ignored or the optical sensor 112 can be turned off. When the motion sensor 128 senses movement of the hearing device 100, the hearing device 100 transitions to the nominal power mode, in which case the optical sensor 112 operates to detect proximity of the hearing device 100 to the user's ear.

A pair of hearing devices 100 can be configured to support streaming via the communication device 124 of the hearing devices 100 and an external content source. When a first hearing device 100 is removed from one of the user's ear while the second hearing device 100 is deployed in the other ear of the user, the stream is paused in the second hearing device 100. When the first hearing device 100 is placed back in the user's ear, the streaming is continued. When both hearing devices 100 are removed from the user's ears while operating a low power mode, routing of phone calls, alarms, and notifications to the hearing devices 100 is prevented or discontinued.

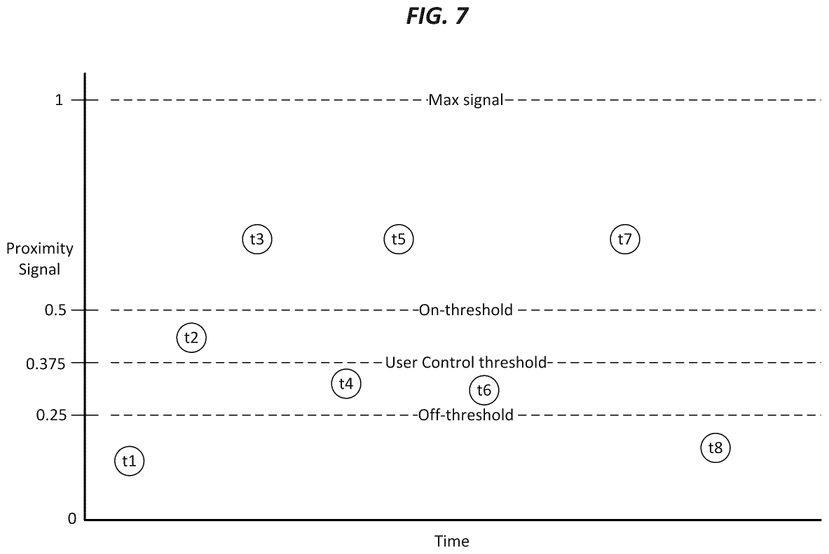

FIG. 7 graphically illustrates a user control feature implemented by the hearing device 100 in accordance with any of the embodiments. The user control feature can be implemented to supplement or replace the switch actuator 104 shown in FIGS. 1 and 2. The user control feature is implemented by the controller 130 in cooperation with the PMIC 126 and optical sensor 112 of the hearing device 100.

In general terms, the controller 130 is configured to operate the hearing device 100 in a nominal power mode in response to a proximity signal, generated by the optical sensor 112, exceeding an On-threshold. The controller 130 is also configured to operate the hearing device 100 in a low power mode in response to the proximity signal falling below an Off-threshold lower than the On-threshold. The controller 130 is further configured to interpret changes of the proximity signal relative to a User Control threshold due to user movement of the ear or the housing 102 as a user input for controlling the hearing device 100. The User Control threshold is a threshold between the On-threshold and the Off-threshold, and the user control can be activated after the proximity signal exceeds the On-threshold.

According to some embodiments, the User Control threshold is continually being adjusted by the equation: 75% of the current proximity signal level. The controller 130 can evaluate the DC value of the returned proximity signal and, if the value falls below 75% of the DC value, then the user control is activated until it comes back above 75% of the DC value. The current proximity signal level can be smoothed using averaging to remove noise and physiologic signals like heart rate and respiration rate.

The user control feature can be implemented by the user pulling or flicking the pinna which temporarily displaces the housing 102 from its resting position on the ear, causing a significant reduction in the light reflected back to the optical sensor 112. Another approach to implementing the user control is to slightly lift the housing 102 from its resting position on the ear. The temporary reduction in the reflected light sensed by the optical sensor 112 is sufficient to implement a user control function, but not so significant that the controller 130 interprets the temporary reduction in reflected light as a powering-off function.

In FIG. 7, the x-axis is time and the y-axis is the normalized proximity signal that ranges between 0 and 1. At time t1, the proximity signal is below an Off-threshold (e.g., 0.25), in which case the hearing device 100 is not resting on the user's ear (e.g., Off-ear condition) and operates in a low-power mode. In the low-power mode, the radio (e.g., BLE radio) of the hearing device 100 is off, the microphone is off, the motion sensor (e.g., IMU) is off, and the DSP is in sleep mode. At time t2, the proximity signal is above the Off-threshold, above the User Control threshold (e.g., 0.375), and below the On-threshold (e.g., 0.5). Until the proximity signal reaches the On-threshold, the hearing device 100 remains in the low-power mode, even though the proximity signal exceeds the User Control threshold. As such, the user control feature is activated after the proximity signal exceeds the On-threshold.

At time t3, the proximity signal exceeds the On-threshold, in which case the hearing device 100 is resting on the user's ear (e.g., On-ear condition) and operates in a nominal power mode. In the nominal power mode, the components and sensors of the hearing device 100 are powered on and fully functional.

At time t4, the proximity signal falls below the User Control threshold and exceeds the Off-threshold. The proximity signal at time t4 indicates that the user control has been activated by the user, such as by pulling the pinna, which temporarily displaces the housing 102 from its resting position on the ear. Activation of the user control at time t4 results in a reduction of the proximity signal, due to a reduction in reflected light from the ear, from a value above the On-threshold (e.g., at time t3) to a value between the User Control threshold and the Off-threshold. At time t5, the proximity signal returns to a level above the On-threshold, and completes activation of the user control as an input. Detection of a proximity signal below the User Control threshold (and above the Off-threshold), such as at t4, in combination with detection of a proximity signal above the On-threshold, such as at t5, is interpreted by the controller 130 as a user control input.

At time t6, the proximity signal falls below the User Control threshold and exceeds the Off-threshold. The proximity signal at time t6 indicates that the user control has again been activated by the user. Activation of the user control at time t6 results in a reduction of the proximity signal from a value above the On-threshold (e.g., at time t5) to a value between the User Control threshold and the Off-threshold. At time t7, the proximity signal returns to a level above the On-threshold, and completes activation of the user control as an input. Detection of a proximity signal below the User Control threshold (and above the Off- threshold), such as at t6, in combination with detection of the proximity signal above the On- threshold, such as at t7, is interpreted by the controller 130 as a user control input.

At time t8, the proximity signal falls below the Off-threshold, indicating that the hearing device 100 has been removed from the user's ear. In response to the proximity signal falling below the Off-threshold, the hearing device 100 operates in the low power mode. In the low-power mode, the radio of the hearing device 100 is off, the microphone is off, the motion sensor is off, and the DSP is in sleep mode. In some implementations, the hearing device 100 transitions from the nominal power mode to the low power mode after a predetermined duration of time (e.g., 10 minutes) of non-movement of the hearing device 100, as measured by the motion sensor of the device 100.

In some embodiments, the duration of time between a pair of proximity signals respectively below the User Control threshold and above the On-threshold can be interpreted by the controller 130 of the hearing device 100 as a short duration input or a long duration input. For example, the duration of time between times t4 and t5 can be interpreted as a short duration input, whereas the duration of time between times t6 and t7 can be interpreted as a long duration input. In other embodiments, the duration of time between a pair of proximity signals respectively below the User Control threshold and above the On-threshold can be interpreted by the controller 130 of the hearing device 100 as a short duration input, a medium duration input, or a long duration input. User control inputs of different durations can correspond to different user inputs for controlling the hearing device 100.

It is noted that the On-threshold, the Off-threshold, and/or the User Control threshold can be adjusted automatically or manually. As previously discussed, the User Control threshold can be automatically and continually adjusted by the equation: 75% of the current proximity signal level. When operating in the nominal power mode, the hearing device 100 can record the proximity signal and automatically adjust the On-threshold based on the recording. For example, the On-threshold can be adjusted to an average of the proximity signal recorded over a specified duration (e.g., 60 to 120 seconds) while operating in the nominal power mode. The specified duration should exceed the average time it takes a user to position the hearing device 100 behind/in the ear. Adjustment of the On-threshold can be limited to avoid a scenario where the hearing device 100 turns on without being deployed on/in the user's ear.

In a similar manner, the hearing device 100 can record the proximity signal when operating in an off-ear power mode, and automatically adjust the Off-threshold based on the recording. For example, the Off-threshold can be adjusted to an average of the proximity signal recorded over a specified duration (e.g., 60 to 120 seconds) while operating in the off-ear low power mode.

It is further noted that, in some implementations, a ratio of reflected light from two or more wavelengths of light emitted by the optical sensor 112 can be used as a threshold.

Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed subject matter may be practiced other than as explicitly described herein.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The terms “connected” or “coupled” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality.

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure.

Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

What is claimed is:

1. A receiver-in-canal (RIC) hearing device, comprising:
a housing configured for deployment behind an ear of a user of the hearing device;
a power source, audio components, a communication device, and a controller respectively disposed in the housing;
a cable assembly comprising a cable, a receiver disposed at a distal end of the cable, and a connector disposed at a proximal end of the cable and configured to attach to the housing; and
an optical sensor disposed in the connector and configured to generate a proximity signal based on proximity of the optical sensor to skin at or near the user's ear;
wherein the controller is configured to operate the hearing device in a nominal power mode in response to the proximity signal exceeding a threshold and to operate the hearing device in a low power mode in response to the proximity signal falling below the threshold.

2. The hearing device according to claim 1, wherein the controller operates the hearing device in the low power mode in response to the proximity signal falling below the threshold for a specified duration of time.

3. The hearing device according to claim 1, comprising a motion sensor disposed in the housing, wherein the controller is configured to operate the hearing device in the low power mode in response to the proximity signal falling below the threshold and the motion sensor indicating an absence of housing motion for a specified duration of time.

4. The hearing device according to claim 1, wherein the audio components communicate a first audio output to the receiver in response to the proximity signal exceeding the threshold and communicates a second audio output or no audio output to the receiver in response to the proximity signal falling below the threshold.

5. The hearing device according to claim 1, wherein the connector comprises:
an interface configured to communicatively couple the optical sensor and the controller; and
power and ground contacts arranged to couple with power and ground contacts of the housing.

6. The hearing device according to claim 1, wherein the optical sensor is configured to sense a heart rate of the user.

7. The hearing device according to claim 1, wherein the optical sensor is configured to sense blood oxygen saturation of the user.

8. The hearing device according to claim 1, wherein:
the optical sensor comprises an infrared (IR) emitter and a photodetector sensitive to IR light; and
the connector comprises material substantially transparent to IR light.

9. The hearing device according to claim 1, wherein:
the optical sensor comprises:
an infrared (IR) emitter;
one or more visible light emitters;
photodetectors sensitive to IR light and one or more visible light wavelengths; and
the connector comprises material substantially transparent to IR light and the one or more visible light wavelengths.

10. The hearing device according to claim 9, wherein the controller is configured to control the one or more visible light emitters as visible indicators to the user.

11. The hearing device according to claim 1, wherein the optical sensor operates at a first update rate when in the low power mode and a second update rate when in the nominal power mode, wherein the second update rate is faster than the first update rate.

12. The hearing device according to claim 1, wherein the power source comprises a lithium- ion battery coupled to a charge pump, a buck converter, a boost converter, or a buck/boost converter.

13. The hearing device according to claims 1, wherein:
the power source comprises a zinc-air battery coupled to a charge pump or a boost converter; and
the optical sensor is coupled to the charge pump or the boost converter.

14. The hearing device according to claim 1, wherein the controller is configured to interpret changes of the proximity signal relative to the threshold due to user movement of the ear or the housing as a user input for controlling the hearing device.

15. The hearing device according to claim 14, wherein the controller is configured to interpret different durations of proximity signal changes as different user inputs.

16. The hearing device according to claim 15, wherein the different durations of proximity signal changes comprise a short duration change and a long duration change.

17. The hearing device according to claim 15, wherein the different durations of proximity signal changes comprise a short duration change, a medium duration change, and a long duration change.

18. The hearing device according to claim 1, comprising an additional optical sensor disposed in the receiver and configured to generate an additional proximity signal based on proximity of the additional optical sensor to skin of the user's ear canal;
wherein the controller is configured to operate the hearing device in the nominal power mode in response to the proximity signal and the additional proximity signal exceeding the threshold and to operate the hearing device in a low power mode in response to the proximity signal and the additional proximity signal falling below the threshold.

19. The hearing device according to claim 1, wherein the controller is configured to:
operate the hearing device in the nominal power mode in response to the proximity signal exceeding an On-threshold; and
operate the hearing device in the low power mode in response to the proximity signal falling below an Off-threshold lower than the On-threshold.

20. The hearing device according to claim 1, wherein the controller is configured to:
operate the hearing device in the nominal power mode in response to the proximity signal exceeding an On-threshold;
operate the hearing device in the low power mode in response to the proximity signal falling below an Off-threshold lower than the On-threshold; and
interpret changes of the proximity signal below a User Control threshold due to user movement of the ear or the housing as a user input for controlling the hearing device, wherein the User Control threshold is between the On-threshold and the Off-threshold and is active after the proximity signal exceeds the On-threshold.

21. A cable assembly configured to attach to a housing of a receiver-in-canal (RIC) hearing device, the cable assembly comprising:
a cable;
a receiver disposed at a distal end of the cable; and
a connector disposed at a proximal end of the cable and configured to attach to the housing of the hearing device, the connector comprising:
an optical sensor configured to generate a proximity signal based on proximity of the optical sensor to skin at or near an ear of a user of the hearing device;
an interface configured to communicatively couple the optical sensor with a controller of the hearing device; and
power and ground contacts configured to couple with power and ground contacts of the hearing device housing;
wherein the connector comprises material substantially transparent to light generated by the optical sensor.

22. The cable assembly according to claim 21, wherein the optical sensor is configured to sense a heart rate of the user.

23. The cable assembly according to claim 21, wherein the optical sensor is configured to sense blood oxygen saturation of the user.

24. The cable assembly according to claim 21, wherein:
the optical sensor comprises an infrared (IR) emitter and a photodetector sensitive to IR light; and
the connector comprises material substantially transparent to IR light.

25. The cable assembly according to claim 21, wherein:
the optical sensor comprises:
an infrared (IR) emitter;
one or more visible light emitters;
photodetectors sensitive to IR light and one or more visible light wavelengths; and
the connector comprises material substantially transparent to IR light and the one or more visible light wavelengths.
